(19) United States
(12) Patent Application Publication
(10) Pub. No.: US 2006/0259092 A1
(43) Pub. Date: Nov. 16, 2006

(54) Contrasted components for a medical device

(76) Inventors: Scott Spadgenske, Fridley, MN (US); Scott Vanderlinde, Plymouth, MN (US); John A. Dyjach, Circle Pines, MN (US); Kathryn Ann Arnold, Carmel, IL (US)

Correspondence Address: SCHWEGMAN, LUNDBERG, WOESSNER & KLUTH, P.A., P.O. BOX 2938, MINNEAPOLIS, MN, 55402, US

(21) Appl. No.: 11/129,070

(22) Filed: May 13, 2005

(51) Int. Cl. A61N 1/375

(52) U.S. Cl. 607/37

(57) ABSTRACT

This document discusses, among other things, contrasted components for a medical device, and methods of making and using contrasted components. An example device includes a header including an opening configured to receive a set screw and a plug, and an insulative component, such as a plug or sleeve, that is insertable in the opening over the set screw. The insulative component has a visible exterior surface that is visually contrasted from a portion of the header proximate the opening. An example method includes forming from a first material a medical device header block having an opening configured to receive a plug, adding a marker to the first material or a second material, and forming from the second material a plug insertable in the opening. The marker in the second material creates or enhances a visual difference between the first material and the second material.

### TECHNICAL FIELD

The present subject matter relates to components for medical devices. More particularly, it pertains to contrasted components for a medical device.

### BACKGROUND

Some medical devices include leads that extend into the body. Implantable pacers and defibrillators, for example, typically include at least one lead that extends around or into the heart. A medical device such as a pacer or defibrillator typically has a header or connector block, to which one or more leads are coupled. In one configuration, a lead is inserted into a receptacle in the header, and a set screw is inserted through a second opening in the header and engaged in the header against the lead to hold the lead in place. In some instances, a lead is secured by two or three set screws. In devices that couple to multiple leads, up to six or more set screws are engaged in a header block.

In some devices, a plug is inserted over a set screw. Some plugs include a slit through which a wrench can be inserted to reach a set screw beneath the seal plug. In an example, a plug is made of silicone. The silicone plug has an elastic quality that allows it to accommodate a wrench that is inserted through the slit, and then re-seal after the wrench is removed. Improved medical device components are needed.

### SUMMARY OF THE INVENTION

An example device includes an implantable housing, a pulse generator coupled to the implantable housing, and a header coupled to the implantable housing and electrically coupled to the pulse generator. The header includes a lead receptacle configured to receive a lead connector and an opening intersecting with the lead receptacle. The example device also includes a set screw insertable in the opening in the header and configured to engage a lead connector inserted in the lead receptacle, and an insulative component insertable in the opening in the header and configured to seal against the header. The insulative component includes a marker that contrasts the insulative component from the header.

Another example implantable medical device includes an implantable structure including an opening configured to receive a set screw, and a plug insertable in the opening with the set screw. The plug includes a marker that contrasts the plug from the implantable structure.

Another example device includes an implantable housing, a pulse generator coupled to the implantable housing, and a header coupled to the implantable housing and electrically coupled to the pulse generator. The header includes a lead receptacle configured to receive a lead connector and an opening intersecting with the lead receptacle. The device also includes a set screw insertable in the first opening in the header and configured to engage a lead connector inserted in the lead receptacle, an insulative component insertable in the first opening in the header and configured to seal against the header, and a visual indicator of the presence of the insulative component in the first opening.

An example method includes adding a marker to a first insulative material or a second insulative material, the marker changing the appearance of the material. The method also includes forming from the first insulative material an implantable medical device header block having an opening, and forming from the second insulative material a component insertable in the opening in the header block. The marker in the first material or second material contrasts the insulative component from the header block.

### BRIEF DESCRIPTION OF THE DRAWINGS

FIG. 1 is an illustration of an example of an implantable device that has leads extending into a heart.

FIG. 2A is a perspective view of a medical device header and leads, screws, and plugs that are insertable into the header.

FIG. 2B is a perspective view of the medical device header of FIG. 2A with leads, screws, and plugs assembled into the header.

FIG. 3A is a front view of a header and a plug that is contrasted from a header.

FIG. 3B is a partial side view of a screw, a plug, and a lead connector in a header block.

FIG. 4A is a perspective view of a medical device header, leads, and screws that are insertable into the header.

FIG. 4B is a perspective view of the medical device header of FIG. 4A with leads and screws assembled into the header.

FIG. 5 is a cross-sectional view of a screw and a plug in a header block.

FIG. 6 is a cross-sectional view of a screw and an insulative sleeve in a header block.

FIG. 7 is a cross-sectional view of an example screw and sleeve in a header block.

FIG. 8 is a cross-sectional view of an example of screw in a header block.

FIG. 9 is a flow chart that illustrates a method of visually differentiating a component from surrounding structure.

### DETAILED DESCRIPTION

The following detailed description includes references to the accompanying drawings, which form a part of the detailed description. The drawings show, by way of illustration, specific embodiments in which the invention may be practiced. These embodiments are also referred to herein as “examples.” The drawings and following detailed description is not to be taken in a limiting sense, and the scope of the present invention is defined by the appended claims and their equivalents.

Overview

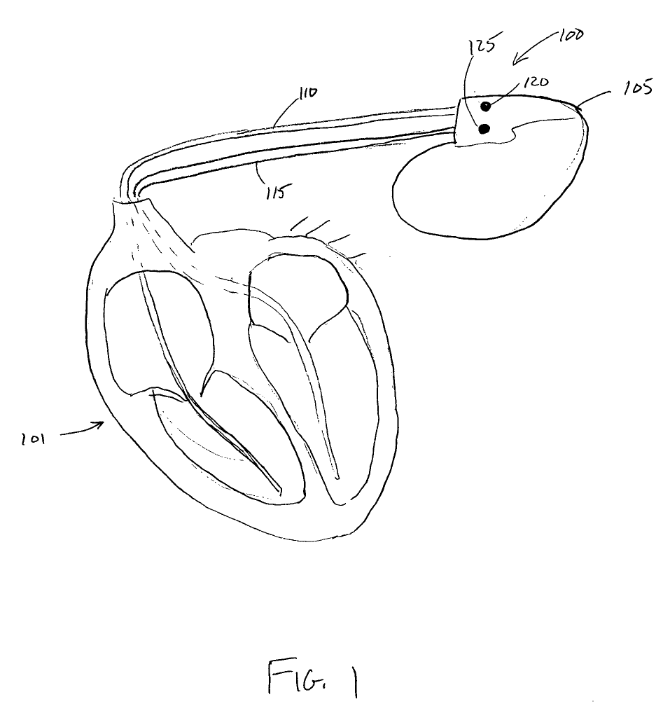

An insulative component, such as plug, is visually differentiated from an implantable structure, such as a medical device header, into which the component is inserted. An example medical device including a header block and contrasting plugs is shown in FIG. 1. In an example, the header block includes polyurethane or epoxy and the plug includes silicone and a marker to contrast the plug from the header block. In one example, the entire plug is contrasted from the header. In another example, only a visible external surface of the plug is contrasted from the header.

In an example, contrasting a component such as a plug from the surrounding structure facilitates location of the component in the device during a medical procedure, such as an implant or explant. In another example, contrasting a component from surrounding structure facilitates verification that a component is present in an opening in the header block. In a device that contains multiple set screws and multiple plugs, for example, it can be helpful to visually verify that the plugs have been assembled into the device, and the contrasting components allow the location of the plugs and/or screws to be easily found.

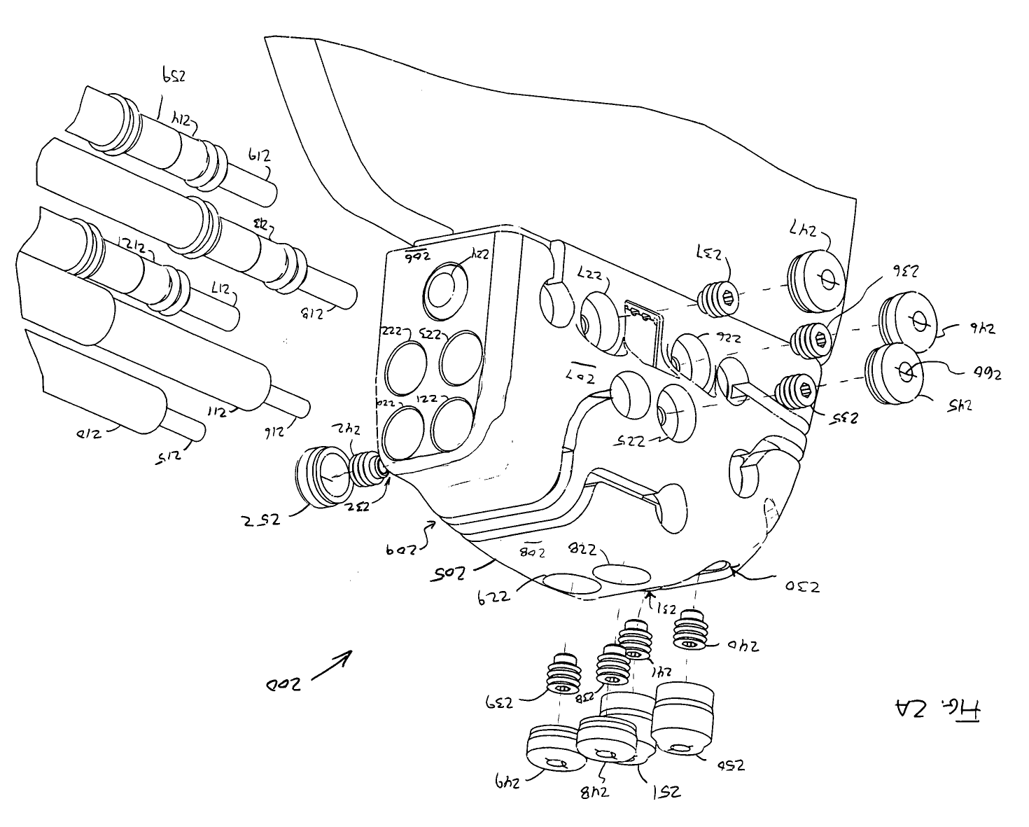

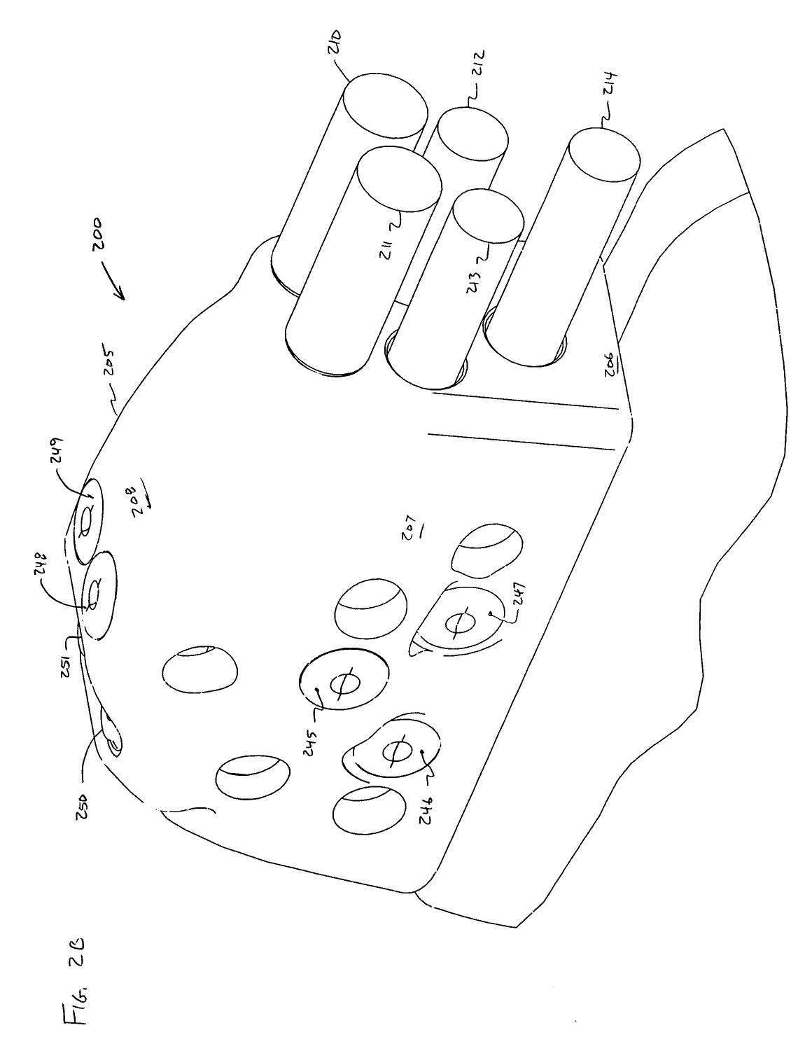

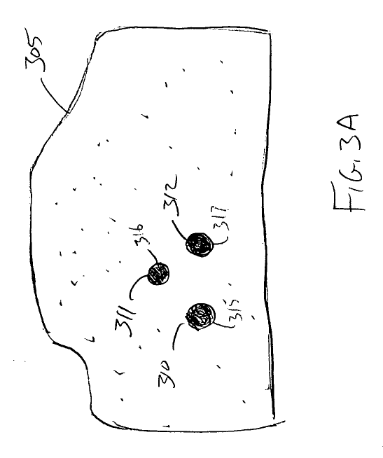

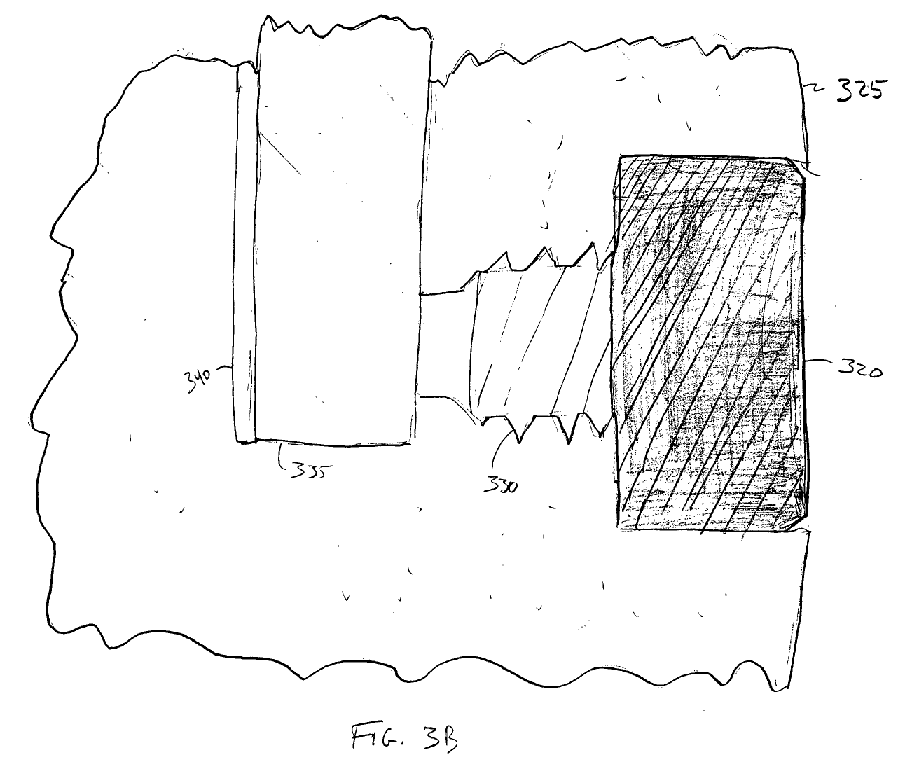

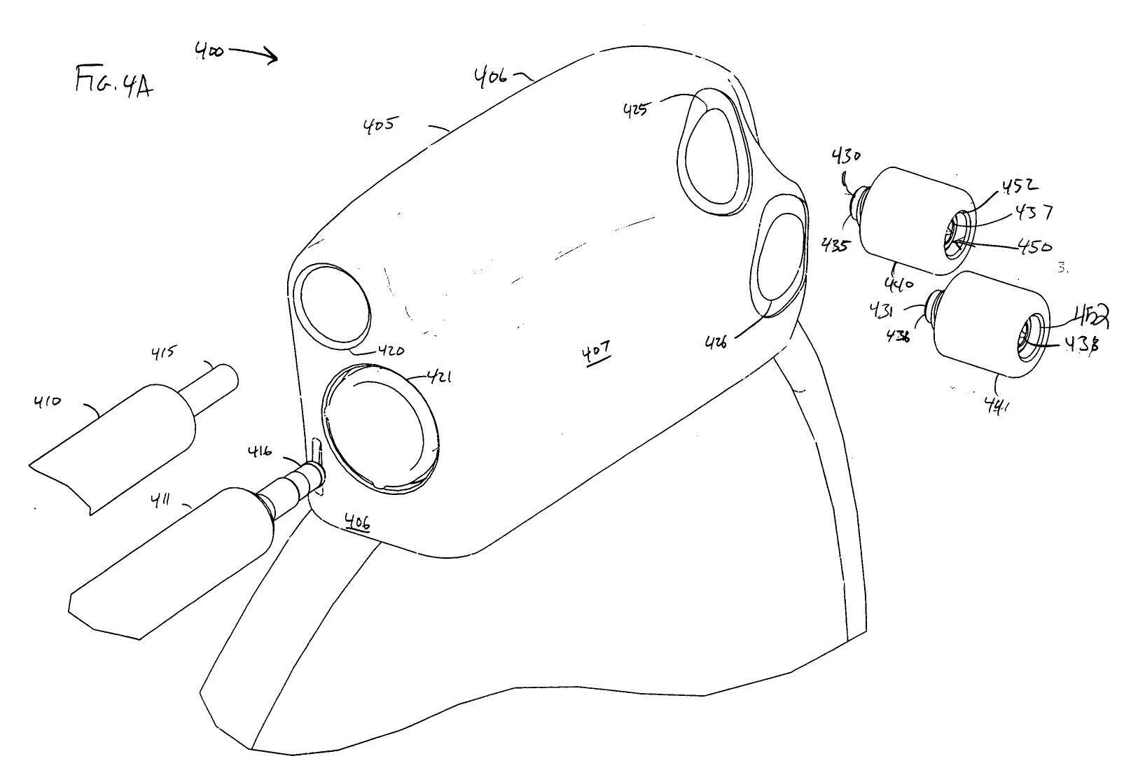

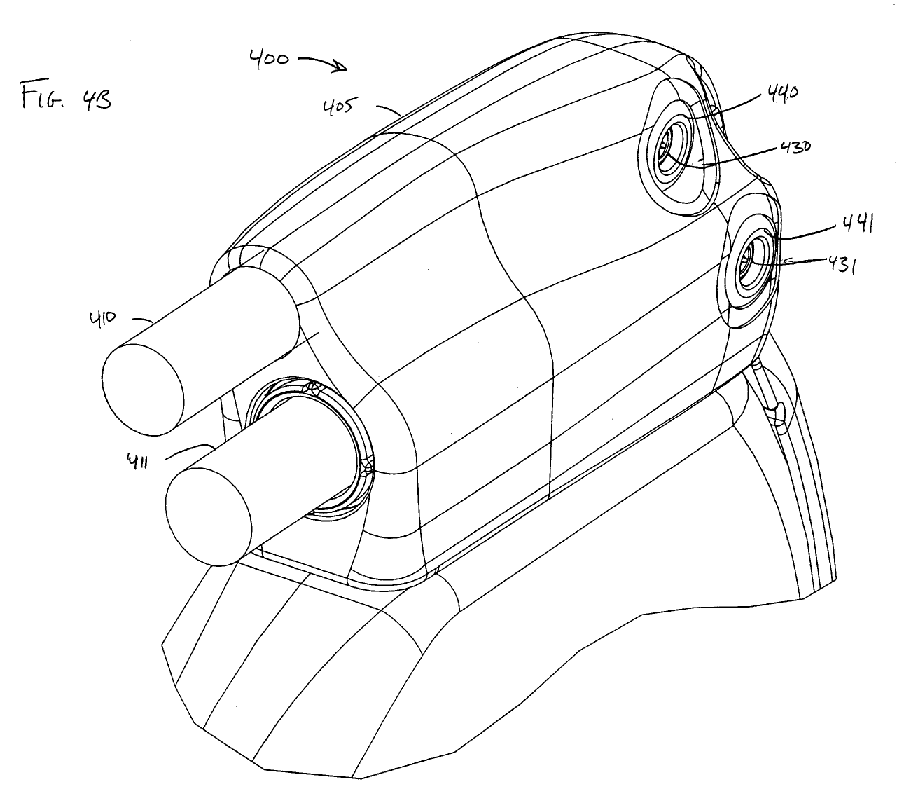

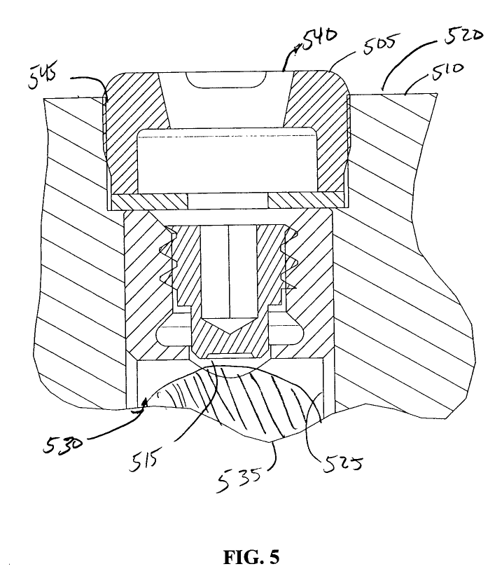

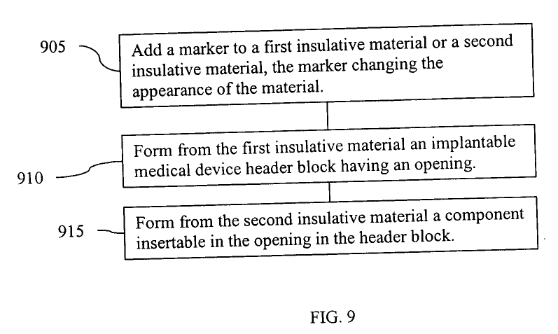

FIGS. 2A-2B show an example configuration of a header, screws insertable in the header, and contrasting plugs insertable over the screws. FIG. 3A shows a front view of a header and plugs that are contrasted from a header. FIG. 3B is a side view that shows a plug that is visible through a header block. FIGS. 4A-4B show an example of a device in which a screw and an insulative sleeve are assembled into a header block, and the screw and/or the sleeve is contrasted from the header block. FIGS. 5-8 show partial cross sections of example connectors and related structure. FIG. 9 is a flow chart that illustrates a method of visually differentiating a component from surrounding structure in a medical device.

Example Techniques for Differentiating Components

In an example, insulative components such as the plugs shown in FIGS. 2A-2B are made from an insulative material such as silicone, and a structure such as the header shown in FIGS. 2A-2B is made from a material that has an appearance similar to silicone, such as polyurethane or epoxy. Polyurethane and epoxy are typically similar in appearance to silicone: Silicone, polyurethane, and epoxy tend to be translucent and mostly colorless. This similarity can make it difficult to visually identify a silicone plug assembled into a polyurethane or epoxy header. For example, in an operating environment, it can be difficult to identify the location of plugs in a device if the silicone material from which the seal plugs are made is similar in color to the header block.

To visually differentiate an insulative component from the surrounding structure, an insulative material is doped, modified, or otherwise processed to modify the appearance of the material. In an example, a marker is added to the insulative component. In an example, the marker is a color agent, such as a pigment, dye, resin, or polymer. In an example, the marker is a pigment such as titanium dioxide (TiO₂). In an example, the color agent is an opaque pigment or dye. In another example, the color agent is a translucent or transparent pigment or dye. In an example, the color agent changes the color, opacity, and/or reflectivity of the insulative component.

In another example, the plugs are made from a fluorescent material. In an example, a marker is added to silicone to make the silicone fluorescent.

In another example, the plugs are made from a material that is excitable to ultra-violet (UV) light. In an example, a UV-excitable marker is added to silicone.

In another example, the marked is added by oxidizing the insulative component material to change the appearance of the material. In an example, a silicone plug material is oxidized.

In another example, aeration and/or oxygenation are used to change the density and color of the material.

In another example, a coating is applied to at least a portion of the insulative component. In an example, a coating is applied to a portion of an outwardly-facing surface of a silicone plug.

In an example, multiple pigments or other markers are used to differentiate among connectors on a header.

In another example, a marker is added to a header to visually differentiate the header from clear, translucent, or colored plugs or screws.

In another example, a clear or translucent plug is assembled over a colored screw or washer. In another example, a bonding agent includes a marker and is bonded to a plug or screw. In an example, the bonding agent is a bead of pigmented adhesive disposed around the perimeter of a clear, translucent, or colored plug.

Example Devices and Systems

Referring now to FIG. 1, an example implantable medical device 100 includes a header 105 and contrasting insulative components 120, 125. In the example shown in FIG. 1, insulative components 120, 125 are plugs. Lead assemblies 110, 115 are coupled to the header 105. In the example shown in FIG. 1, lead assembly 110 extends into the right side of the heart and lead assembly 115 extends into the left side of the heart 101. In an alternative example, the lead assemblies extend on or around the outside of the heart, or on or around other organs or nerve centers where stimulation or sensing is needed.

Each lead assembly 110, 115 includes one or more leads that is coupled to the header 105 with connector, such as a screw, that is inserted through an opening in the header. Connection systems are shown in more detail in FIGS. 2A-2B, 4A-4B, 5, and 6. In the example shown in FIG. 1, plugs 120, 125 are inserted into the openings in the header block over screws. In an example, the plugs 120, 125 create a seal against the header.

The plugs 120, 125, shown in FIG. 1 are contrasted from the header through one of the techniques described above. In an example, the contrast between the plugs and the header facilitates location of the plugs by a surgeon during implant or explant of the device 100.

In an example, titanium dioxide powder, which is typically white, is used as a pigment for silicone. The titanium dioxide powder is mixed with silicone, and the silicon/titanium dioxide powder is molded into a seal plug. The molded seal plug is white. The white seal plug contrasts against a typically transparent or translucent polyurethane header. It is understood that other pigments or other marking techniques can be used.

Referring now to FIG. 2A, an example medical device 200 includes a header 205 and contrasting plugs 245, 246, 247, 248, 249, 250, 251, 252. The header 205 includes a header block and internal electrical connections and electrical circuitry. Header 205 has an end surface 206, front face 207, a top 208, and a back face 209. The end surface 206 includes receptacles 220, 221, 222, 223, 224 into which respective leads 210, 211, 212, 213, 214 can be inserted. The leads 210, 211, 212, 213, 214 include lead connectors, such as pins 215, 216, 217, 218, 219. When the leads are assembled into the header 205, pins 215, 216, 217, 218, 219 on the leads 210, 211, 212, 213, 214 contact the electrical connections on the inside of the header. In an example, pins 215 and 216 connect to a defibrillation circuit, pins 217 and 218 connect to a sensing circuit, and pin 219 connects to a pacing circuit. In an example, two or more of the leads are combined into a lead assembly.

In an example, a lead is coupled to the header with one or more connectors, such as a set screw. In the example shown in FIG. 2A, lead 211 is insertable in opening 221 and connectable to header 205 with screw 238 that is insertable into opening 228 in the top 208 of the header. Screw 238 is engageable on threaded portions of the header. Screw 238 engages the pin 216 on the lead 211. Plug 248 is insertable in the opening 228 over the screw 238. In an example, the plug 248 covers the respective screw 238 and prevents body fluid from traversing the opening 228 and contacting the screws. In an example, the plug prevents electrical conduction from the screw 238 through the opening 228.

The other leads shown in FIG. 2A can also be coupled to the header with screws. Lead 210 is connectable to header 205 with screw 239 that are insertable into opening 229 in the top 208 of the header. Screw 239 engages the pin 215 on the lead 210. Plug 249 is insertable in the opening 229 over the screw 239. Lead 214 is connectable to header 205 with screws 236, 237 that are insertable into openings 226, 227 in the front face 207 of the header. Screw 236 engages the pin 219 on the lead 214 and screw 239 engages a ring portion 259 of the lead. Plugs 246, 247 are insertable in the openings 226, 227 over the screws 236, 237. Leads 213, 212 are connectable to header 205 with respective screws 235, 242 that are insertable into respective openings 225, 232 in the respective front and back faces 207, 209 of the header. Plugs 245, 252 are insertable in the respective openings 225, 232 in the header to cover respective screws. Plugs 250, 251 are insertable in openings 230, 231 over screws 240, 241.

In an example, the plugs 245, 246, 247, 248, 249, 250, 251, 252 include a marker, such as a color agent, oxidized surface or volume, fluorescent or UV-excitable agent, or a coating.

Referring now to FIG. 2B, the medical device of FIG. 2A is shown with leads, screws, and plugs assembled into the header. The leads 210, 211, 212, 213, 214 are inserted into openings 220, 221, 222, 223, 224 in the end of the header. The plugs 245, 246, 247, 248, 249, 250, 251, 252 are inserted in the respective openings 225, 226, 227, 228, 229, 230, 231 over the respective connectors 235, 236, 237, 238, 239, 240, 241, 242.

In an example, one or more of the plugs includes a slit through which a wrench can be inserted. Plug 245, for example, includes a slit 260 (FIG. 2A). In an example, the plug 245 is formed from an elastic material, such as silicone, which can be displaced to accommodate a wrench through the slit. When the wrench is removed, the elastic material returns to its original shape, and the slit closes.

In an example, an exposed outer surface of a component is contrasted from local portions of header block. In FIG. 3A, for example, the outer surfaces 315, 316, 317 of plugs 310, 311, 312 are contrasted from the appearance of header block 305. FIGS. 2A and 2B also show plugs that have an outer surface that is visually differentiated from the header.

In another example, a header is translucent or transparent, and a side portion of a plug or other component is contrasted from the header material, such that the component is visually detectable through the block. In the side view shown in FIG. 3B, for example, seal plug 320 is visible through header block 325. In the example shown in FIG. 3B, a screw 330 and lead 335, and electrical terminal 340 are also visible through the header block. In an example, the visibility of the plug 320 through the header block facilitates confirmation that plugs and/or screws have been assembled into the block.

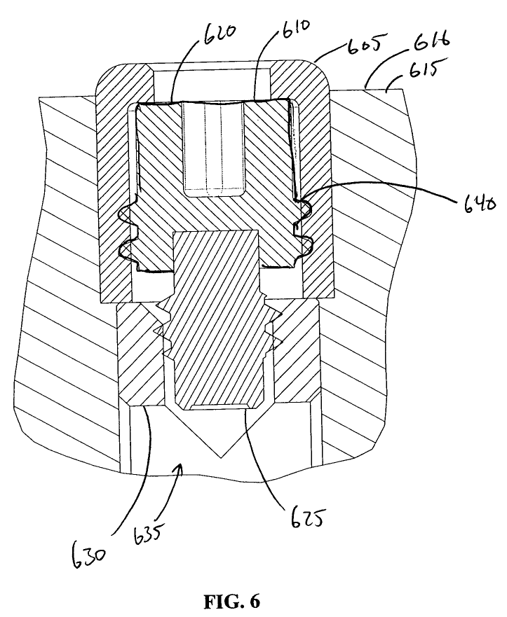

Referring now to FIGS. 4A and 4B, another example of a medical device 400 includes a header 405, a screw 430 and sleeve 440. The sleeve 440 and/or screw are contrasted from the header. Both the screw 430 and the sleeve 440 are insertable an opening 425 in the header 405. The screw is shown inserted into the sleeve in FIG. 4A. The structure of an example screw and sleeve is shown in cross-section in FIG. 6.

Returning to FIG. 4A, the header 405 includes a header block 406 and internal circuitry. The screw 430 includes a threaded shaft 435 and an insulative cap 437 that is coupled to the screw. The cap 437 also includes a driver interface 450 that is configured to receive a driver, such as a wrench. The sleeve 440 includes an orifice 452 through which a wrench can be inserted to turn the screw. The screw 430 is insertable into the sleeve 440. Both the sleeve 440 and screw 430 are insertable into an opening 425 in the header block 406. The threaded shaft 435 engages internal threads in the header. The example in FIG. 4A also includes a second screw 431 that includes a threaded shaft 436 and an insulative cap 438 connected to the threaded shaft. Screw 431 is insertable into sleeve 441. Screw 431 and sleeve 441 are insertable in opening 426 in the header block 406.

Referring again to FIG. 4A, leads 410, 411 are insertable in openings 420, 421 in the header 405. Pins 415, 416 on the leads are shaped and sized to contact electrical connections in the header 405. The threaded shafts 435, 436 are engagable against the pins 415, 416 to secure the pins and leads 410, 411 in the header 405. FIG. 4B shows leads 410, 411 and screws 430, 431 assembled into the header 405.

FIG. 5 is a cross-sectional view of an example system that includes a plug 505 and screw 515 in a header 510. Header 510 includes header block 520 and threaded screw terminal 525. When the screw is turned, it descends into lead receptacle 530 and presses against lead connector 535. The plug 505 includes a slit 540, through which a wrench can be inserted. In an example, the plug is sized to create a press fit in the opening 545 in the header block 520. The plug 505 is contrasted from the header block portion of the header 510. In an example, the plug is formed from silicone that includes a marker that changes the appearance of the silicone.

FIG. 6 shows another example system that includes a sleeve 605 and a screw 610 in a header 615. Header includes header block 616 and connector terminal 630. Screw 610 includes an insulative cap 620 coupled to a threaded shaft 625. In an example, the cap 620 is overmolded onto the shaft 625. In an example, the cap 620 is overmolded onto the shaft 625 in an insert-molding process. The threaded shaft 625 screws into the connector terminal 630. When the threaded shaft 625 is turned, it descends into lead receptacle 635 and contacts a lead connector (not shown). In an example, the insulative cap 620 is over-molded on the threaded shaft 625 in an insert-molding process. The insulative cap 620 includes one or more ribs 640 that press against the sleeve 605. In an example, the sleeve 605 is formed from a compressible material, such as silicone. In an example, the sleeve 605 includes a marker that contrasts the sleeve from the header block portion of the header 615. In another example, the insulative cap 620 includes a marker that contrasts the cap 620 from the header block portion of the header 615. In an example, the cap is formed from a colored polymer. In an example, both the sleeve and the insulative cap include a marker that contrasts the sleeve and the insulative cap from the header block 616. In an example, the insulative cap 620 includes a marker that is different from the marker included in the sleeve 605.

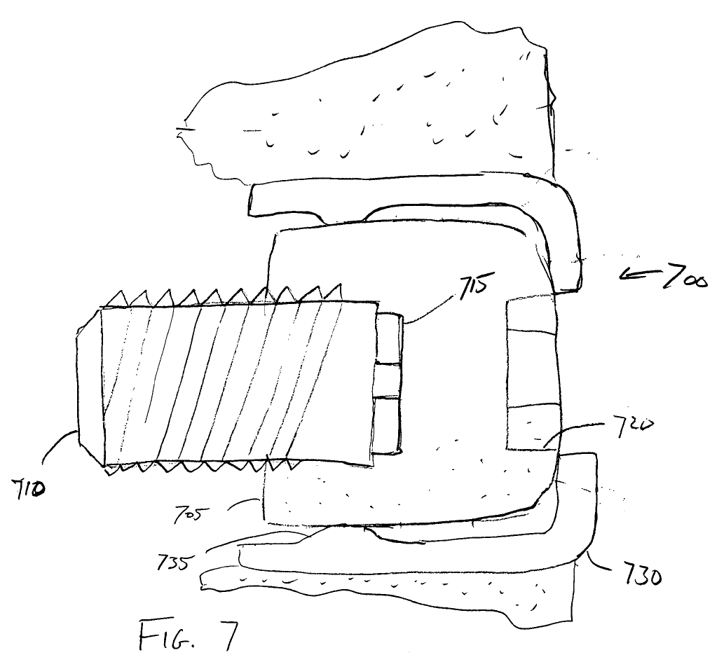

FIG. 7 shows a cross-sectional view of another example screw 700. A cap 705 is connected to a threaded shaft 710 and insertable in a sleeve 730. The cap 705 is formed from a non-conductive material, such as a non-conductive polymer. In an example, the cap 705 is over-molded on the threaded shaft 710. In an example, the threaded shaft includes a structure 715 that facilitates transfer of torque from the cap to the screw. In an example, the structure 715 is cross-shaped. The cap also includes a driver interface 720. The cap is insertable in the sleeve 730. In an example, the sleeve 730 includes a rib 735 that presses against the cap 705 to make a seal. Alternatively the sleeve is configured to make a press fit between the cap 705 and an internal surface of an opening in a header block. In an example, the sleeve 730 includes a marker that contrasts the sleeve from a header or other structure. In another example, the cap includes a marker that contrasts the cap from a header or other structure. In another example, both the sleeve and the cap include markers that contrast the sleeve and cap from a header or other structure.

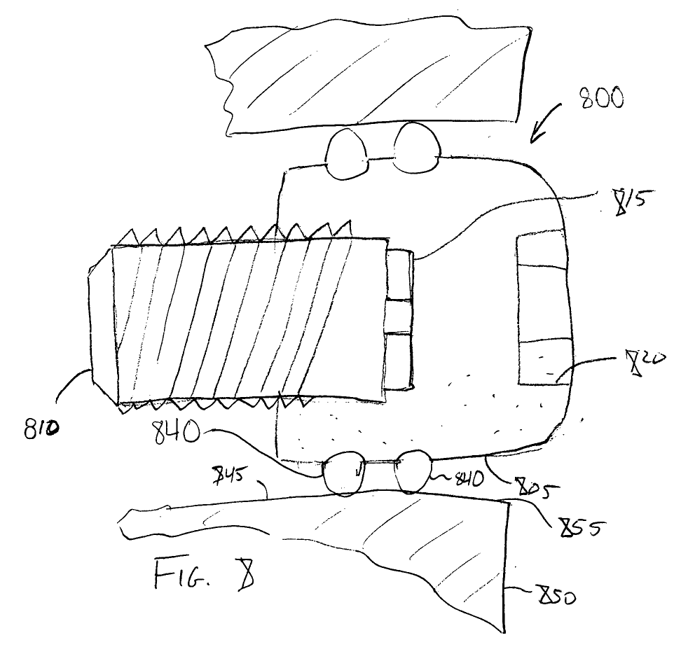

FIG. 8 shows another example screw 800, in which one or more compressible ribs 840 are coupled to a cap 805. Cap 805 is connected to a threaded shaft 810. The cap 805 is formed from a non-conductive material, such as a non-conductive polymer. In an example, the cap 805 is over-molded on the threaded shaft 810. In an example, the threaded shaft includes a structure 815 that facilitates transfer of torque from the cap to the screw. In an example, the structure 815 is cross-shaped. The cap also includes a driver interface 820. Compressible ribs 840 are coupled to the cap. In an example, the compressible ribs are O-rings. Compressible ribs 840 press against an inner surface 845 of an opening 855 in a header 850 to form a seal. The cap 805 includes a marker to contrast the cap 805 from the header 850.

FIG. 9 is a flow chart that illustrates a method of differentiating a component from surrounding structure. At 905, a marker is added to a first insulative material or a second insulative material. The marker changes the appearance of the material to which it is added. At 910, an implantable medical device header block is formed from the first material. The implantable medical device header block includes an opening, into which a screw and seal plug can be inserted, for example. At 915, a component, such as a seal plug or sleeve, is formed from the second insulative material. In an example forming a component from the second material includes insert molding a cap onto a threaded shaft. The marker in the first material or second material contrasts the insulative component from the header block. In an example, the first material is polyurethane or epoxy, and the second material is silicone. In an example, adding a marker includes oxidizing at least a portion of the first or second insulative material, such as oxidizing silicone, for example. In an example, adding a marker includes adding a color agent. In an example, adding a marker changes the opacity, reflectivity and/or color of the first or second material. In an example, a marker is added to both the first material and the second material.

It is to be understood that the above description is intended to be illustrative, and not restrictive. Since many embodiments of the invention can be made without departing from the spirit and scope of the invention, the invention resides in the claims hereinafter appended.

What is claimed is:

1. A device comprising:
an implantable housing;
a pulse generator coupled to the implantable housing;
a header coupled to the implantable housing and electrically coupled to the pulse generator, the header including a lead receptacle configured to receive a lead connector and an opening intersecting with the lead receptacle;
a set screw insertable in the opening in the header and configured to engage a lead connector inserted in the lead receptacle; and
an insulative component insertable in the opening in the header and configured to seal against the header, the insulative component including a marker that contrasts the insulative component from the header.

2. The device of claim 1, wherein the insulative component is a plug.

3. The device of claim 1, wherein the set screw includes a threaded shaft and an insulative cap coupled to the threaded shaft, and the insulative component is an insulative sleeve insertable between the insulative cap and the header.

4. The device of claim 3, wherein the insulative cap includes a marker that contrasts the insulative cap from the header.

5. The device of claim 1, wherein the insulative component is formed from a material including a color agent that contrasts the insulative component from the header.

6. The device of claim 5, wherein the color agent is a pigment.

7. The device of claim 5, wherein the color agent is a dye.

8. The device of claim 5, wherein the color agent is a resin.

9. The device of claim 5, wherein the color agent is a polymer.

10. The device of claim 5, wherein the color agent is titanium dioxide.

11. The device of claim 1, wherein the header is formed from a translucent material and the marker contrasts the insulative component from the translucent material.

12. The device of claim 1, wherein the header is formed from a transparent material and the marker contrasts the insulative component from the transparent material.

13. The device of claim 1, wherein the header includes an epoxy header block and the insulative component includes silicone.

14. The device of claim 1, wherein the header includes a polyurethane header block and the insulative component includes silicone.

15. The device of claim 1, further comprising a lead assembly including a lead connector insertable in the lead receptacle.

16. A device comprising:
an implantable structure including an opening configured to receive a set screw; and
a plug insertable in the opening with the set screw, the plug including a marker that contrasts the plug from the implantable structure.

17. The device of claim 16, wherein the plug includes a plug body that includes a stock material and a marker added to the stock material.

18. The device of claim 16, wherein the plug includes a plug body and a coating on the plug body, the coating including the marker.

19. The device of claim 16, wherein the plug includes an oxidized portion that includes the marker.

20. The device of claim 19, wherein the plug includes oxidized silicone.

21. The device of claim 16, wherein the marker includes a fluorescent material.

22. The device of claim 16, wherein the marker includes a material that is excitable by ultra-violet light.

23. The device of claim 16, further comprising a set screw and a lead assembly including a lead connector, wherein the implantable structure is a medical device header including a lead receptacle configured to receive the lead connector, and the set screw is insertable in the opening in the header and engageable against the lead connector to secure the lead connector in the header.

24. A method comprising:
adding a marker to at a first insulative material or a second insulative material, the marker changing the appearance of the material;
forming from the first insulative material an implantable medical device header block having an opening; and
forming from the second insulative material a component insertable in the opening in the header block, wherein the marker in the first material or second material contrasts the insulative component from the header block.

25. The method of claim 24, wherein the first material is polyurethane, the second material is silicone, and adding the marker to the silicone or polyurethane contrasts the silicone from the polyurethane.

26. The method of claim 24, wherein the first material is an epoxy, the second material is silicone, and adding the marker to the silicone or epoxy contrasts the silicone from the epoxy.

27. The method of claim 24, wherein adding a marker includes oxidizing at least a portion of the first insulative material or second insulative material.

28. The method of claim 24, wherein adding a marker includes adding a color agent.

29. The method of claim 24, wherein adding a marker changes the opacity of the first insulative material or second insulative material.

30. The method of claim 24, wherein adding a marker changes the reflectivity of the first insulative material or second insulative material.

31. The method of claim 24, wherein adding a marker changes the color of the first insulative material or second insulative material.

32. The method of claim 24, wherein forming from the second insulative material a component insertable in the opening in the header block includes forming a plug configured to form a seal against the header block.

33. The method of claim 24, wherein forming from the second insulative material a component insertable in the opening in the header block includes insert molding a cap on a threaded shaft to form a screw insertable in the opening in the header block.

34. The method of claim 24, wherein forming from the second material a component insertable in the opening in the header block includes forming a sleeve configured to form a seal against a screw that is insertable in the opening in the header block.

35. A device comprising:
an implantable housing;
a pulse generator coupled to the implantable housing;
a header coupled to the implantable housing and electrically coupled to the pulse generator, the header including a lead receptacle configured to receive a lead connector and an opening intersecting with the lead receptacle;
a set screw insertable in the first opening in the header and configured to engage a lead connector inserted in the lead receptacle; and
a first insulative component insertable in the first opening in the header and configured to seal against the header; and
a visual indicator of the presence of the insulative component in the first opening or a location of the opening.

36. The device of claim 35, wherein the visual indicator includes a washer inserted in the opening between the insulative component and the header.

37. The device of claim 35, wherein the visual indicator includes cavities in the insulative component, the cavities providing the insulative component with an appearance that contrasts with the header.

38. The device of claim 35, wherein the visual indicator of the presence of the insulative component includes an adhesive applied to the insulative component and the header, the adhesive including a marker that contrasts the adhesive from the header.

39. The device of claim 35, wherein the header further comprises a second opening, the device further comprising a second insulative component insertable in the second opening and including a marker that visually differentiates a location of the second opening from the location of the first opening.
